(19) United States
(12) Patent Application Publication
(10) Pub. No.: US 2026/0114778 A1
(43) Pub. Date: Apr. 30, 2026

(54) ECG PATCH WITH HAIR GROWTH INHIBITOR

(71) Applicant: KONINKLIJKE PHILIPS N.V., EINDHOVEN (NL)

(72) Inventor: Victor Martinus Gerardus van Acht, Eindhoven (NL)

(21) Appl. No.: 19/363,890

(22) Filed: Oct. 21, 2025

Related U.S. Application Data
(60) Provisional application No. 63/713,723, filed on Oct. 30, 2024.

(51) Int. Cl. A61B 5/257; A61B 5/00; A61B 5/265; A61B 5/282

(52) U.S. Cl. CPC A61B 5/257; A61B 5/265; A61B 5/282; A61B 5/6823; A61B 5/6832; A61B 2560/0468; A61B 2562/0215; A61B 2562/12; A61B 2562/14

(57) ABSTRACT

A method for manufacturing a monitoring unit, comprising: providing one or more electrodes of the monitoring unit, wherein the one or more electrodes are configured to measure one or more physiological signals of the patient; providing a signal processor configured to receive the one or more physiological signals; and adding a hair-growth inhibitor to at least a portion of the one or more electrodes.

### CROSS-REFERENCE TO RELATED APPLICATIONS

This patent application claims the priority benefit under 35 U.S.C. § 119(e) of U.S. Provisional Application No. 63/713,723, filed on October 30, 2024, the contents of which are herein incorporated by reference.

### FIELD OF THE INVENTION

The present invention pertains to methods, devices, and units for inhibiting hair growth and in particular, inhibiting hair growth for a physiological patch.

### BACKGROUND OF THE INVENTION

Electrocardiography (ECG or EKG) is a technique commonly used to record the heart’s electrical activity through repeated cardiac cycles in order to monitor a patient's heartbeat for cardiac abnormalities. Employing this technique, several electrodes are attached to the outer surface of the skin at certain places on the patient’s torso and extremities in order to monitor the electrical activity of the heart. These electrodes detect the small electrical changes that are a consequence of cardiac muscle depolarization followed by repolarization during each cardiac cycle. Changes in the normal ECG pattern occur in numerous cardiac conditions, including cardiac rhythm disturbances, such as atrial fibrillation and ventricular tachycardia, inadequate coronary artery blood flow, such as myocardial ischemia and myocardial infarction, and electrolyte disturbance, such as hypokalemia and hyperkalemia. The electrodes are connected by lead wires to an external device, which records the electrical activity of the heart over a period of time as detected by the electrodes. The data recording produced by the ECG technique is an electrocardiogram.

Conventional electrocardiograms employ ten electrodes for measuring the electrical activity of the heart, where each electrode is placed on a patient at a particular location within some tolerance. From these ten electrodes, twelve leads (i.e., potential differences) are measured and/or derived. For example, a right leg electrode (“RL”) can serve as a ground for the other electrodes, a first lead (Lead I) is measured from a right arm electrode (“RA”) to a left arm electrode (“LA”), a second lead (Lead II) is measured from the right arm electrode RA to a left leg electrode (“LL”), a third lead (Lead III) is measured from a left arm electrode LA to the left leg electrode LL, while the other nine leads include three augmented limb leads derived from combinations of Leads I-III and six chest leads derived from potential differences measured using six electrodes placed on the patient’s chest at predetermined positions. Other conventional electrocardiograms include one, four, or five leads from a set of two, three, five or six electrodes. An electrocardiogram produced through these various arrangements is a valuable, non-invasive, diagnostic and monitoring tool that records the heart’s electrical activity as waveforms and can detect various heart conditions.

These solutions are traditionally offered in Holter and mobile telemetry propositions such that patient wear an adhesive patch with embedded electrodes together with an electronic sensor for 3 to 30 days to collect diagnostic data. However, during this time a patient’s hair might regrow and interfere with signal quality, cause the electrodes to fall off early, and/or cause pain for the patient during removal. Proper electrode attachment is essential to measure the heart’s electrical activity accurately and reliably diagnose and interpret cardiac abnormalities or arrhythmias because the standards for normal and abnormal conditions are based on those standard recordings.

### SUMMARY OF THE INVENTION

There is thus a continued need to minimize hair growth of a patient to keep the electrodes attached firmly to the patient, ensure good signal quality, and make removal of the electrodes less painful. This results in significant improvements in diagnosis and care of a patient as well as overall comfort. Accordingly, it is an object of the present invention to provide a patient-portable remote monitoring unit that overcomes the shortcomings of conventional patient-portable remote monitoring units. This object is achieved according to one embodiment of the present invention by providing a patient-portable remote monitoring unit that includes a hair-growth inhibitor.

It is yet another object of the present invention to provide a method of manufacturing the patient-portable remote monitoring unit that does not suffer from the disadvantages associated with conventional techniques. This object is achieved by providing a method of manufacturing that includes providing a hair-growth inhibitor.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

According to an embodiment of the present disclosure, a method for manufacturing a monitoring unit is provided. The method comprises providing one or more electrodes of the monitoring unit, wherein the one or more electrodes are configured to measure one or more physiological signals of the patient, providing a signal processor configured to receive the one or more physiological signals, and adding a hair-growth inhibitor to at least a portion of the one or more electrodes.

In an aspect, the one or more electrodes comprises an adhesive portion and a gel portion.

In an aspect, adding the hair-growth inhibitor to at least a portion of the one or more electrodes comprises adding the hair-growth inhibitor to the adhesive portion, wherein the adhesive portion comprises an adhesive configured to hold the one or more electrodes of the patient-portable remote monitoring unit into place on a chest of a patient.

In an aspect, adding the hair-growth inhibitor to at least a portion of the one or more electrodes comprises adding the hair-growth inhibitor to the gel portion, wherein the gel portion comprises a material and a gel and is configured to form an electrical interface to measure the physiological signals.

In an aspect, the gel is a silver chloride solution.

In an aspect, adding the hair-growth inhibitor to at least a portion of the one or more electrodes comprises adding the hair-growth inhibitor to the adhesive portion and the gel portion.

In an aspect, the method further comprises creating inhibited electrodes.

In an aspect, the method further comprises applying a protective covering layer to the inhibited electrodes.

In an aspect, adding the hair-growth inhibitor to an external gel, wherein the external gel is added to the one or more electrodes of the patient-portable remote monitoring unit prior to being placed on a chest of a patient.

According to another embodiment of the present disclosure, a monitoring unit to be worn by a patient is provided. The unit comprises one or more electrodes, comprising an adhesive portion of the one or more electrodes, wherein the adhesive portion of the one or more electrodes holds the one or more electrodes into place on a chest of the patient and comprises a hair-growth inhibitor, and a gel portion of the one or more electrodes, wherein the gel portion comprises a material and a gel and is configured to form an electrical interface to measure the physiological signals, and a signal processor configured to receive the one or more physiological signals.

In an aspect, the gel is a silver chloride solution.

In an aspect, the unit further comprises an external gel, wherein the external gel is added to the one or more electrodes of the patient-portable remote monitoring unit prior to being placed on a chest of a patient.

According to another embodiment of the present disclosure, a monitoring unit to be worn by a patient is provided. The unit comprises one or more electrodes, comprising an adhesive portion of the one or more electrodes, wherein the adhesive portion of the one or more electrodes holds the one or more electrodes into place on a chest of the patient, and a gel portion of the one or more electrodes, wherein the gel portion comprises a material and a gel and is configured to form an electrical interface to measure the physiological signals, and comprises a hair-growth inhibitor, and a signal processor configured to receive the one or more physiological signals.

In an aspect, the chest is pre-treated with an additional hair-growth inhibitor.

In an aspect, the gel is a silver chloride solution.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIGS. 1A, 1B, and FIG. 1C are diagrams illustrating inhibiting hair growth according to aspect of the present disclosure.

FIG. 2 illustrates a flowchart of the system for inhibiting hair growth, in accordance with an embodiment.

### DETAILED DESCRIPTION OF EMBODIMENTS

The present disclosure is related to methods, devices, and units for inhibiting hair growth. As noted above, there is a need for solutions related to inhibiting hair growth with respect to electrodes of Holter and mobile telemetry propositions.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

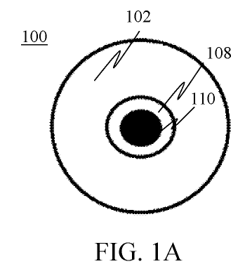

Turning to FIG. 1A, electrode 100 configured to inhibit hair growth on patient 105 is illustrated in accordance with certain aspects of the present disclosure. As shown in FIG. 1A, electrode 100 includes adhesive portions 102, , ring 108, and center 110. In some embodiments, center 110 is a conductive material, such as silver. In some embodiments, center 110 is covered by a gel (e.g., silver chloride solution) that collectively creates a good electrical contact to a patient’s body to measure physiological signals, such as ECG signals. In some embodiments, adhesive portions 102are configured to stick electrode 100 to a patient’s body. In some embodiments, ring 108 surrounds center 110 such that the gel might extend onto ring 108. In some embodiments, hair-growth inhibitor is added to one or more of adhesive portions 102, ring 108, or center 110. In some embodiments, the hair-growth inhibitor prevents hair re-growth while the electrode is being worn. This is beneficial and allows for the improvement of physiological signal quality since the less hair present allows for a better physiological signal measurement. Further, the hair-growth inhibitor allows for a less painful electrode removal for the patient. Still further, the hair-growth inhibitor allows for the electrode(s) to stay on as hair re-growth can cause electrodes to fall off. In some embodiments, and depending on the duration the patch that includes the electrode is going to be worn, the concentration or dosage of the ingredients may be adapted.

Turning to FIG. 1B, electrode 100 can be placed on chest 115 of patient 105. In some embodiment, chest 115 is pre-treated for hair growth inhibiting. For instance, chest 115 may be shaved prior to applying electrode 100. In some embodiments, an external gel with a hair-growth inhibitor is applied to chest 115 prior to placing electrode 100. In some embodiments, the electrodes are part of a monitoring unit that is worn by the user for a long period of time outside of a hospital setting (e.g., patient-portable). In some embodiments, the electrodes are part of a monitoring unit that is worn by the user for a long period of time inside a hospital setting.

Turning to FIG. 1C, electrode 100 can be part of patch 117 which includes signal processor 119. In some embodiments, signal processor 119 is configured to receive and/or analyze the physiological signals to determine an outcome for patient 105.

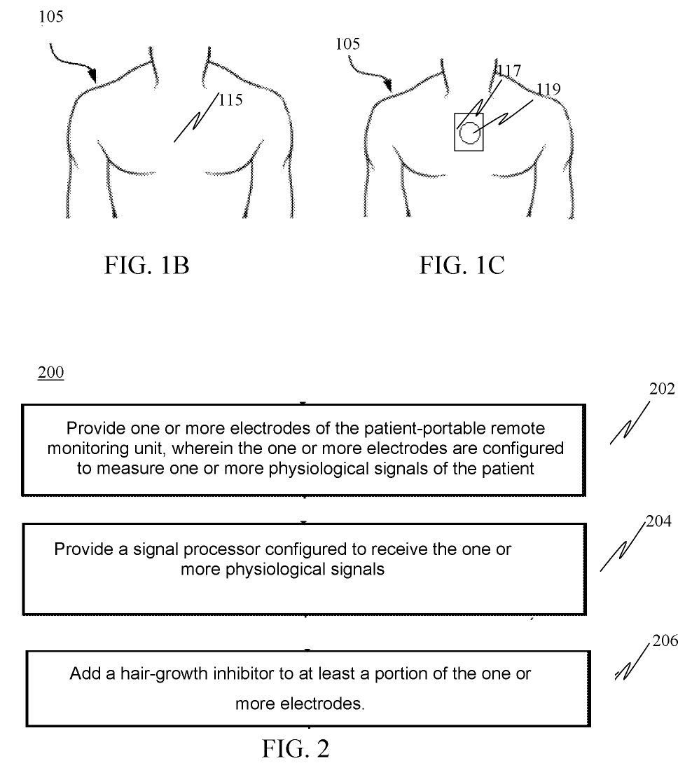

FIG. 2 shows a flowchart 200 for inhibiting hair growth, in accordance with an embodiment. As shown in FIG. 2, flowchart 200 begins with step 202. At step 202, one or more electrodes of the patient-portable remote monitoring unit are provided, wherein the one or more electrodes are configured to measure one or more physiological signals of the patient. For instance, electrode 100 is provided on patch 117 to be applied to chest 115 of patient 105.

As step 204, a signal processor configured to receive the one or more physiological signals is provided. For instance, processor 119 of patch 117 is configured to receive the one or more physiological signals of patient 105.

At step 206, a hair-growth inhibitor is added to at least a portion of the one or more electrodes. For instance, a hair-growth inhibitor is applied to one or more of adhesive portions 102, ring 108, and center 110. In some embodiments, the addition of the hair-growth inhibitor makes electrode 100 an inhibited electrode. In some embodiments, a protective covering is added over the inhibited electrode(s) such that the hair-growth inhibitor is protected until it comes into contact with the patient.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. Other variations to the disclosed embodiments can be understood and effected by those skilled in the art in practicing the claimed invention, from a study of the drawings, the disclosure, and the appended claims. For example, it is to be understood that the present invention contemplates that, to the extent possible, one or more features of any embodiment can be combined with one or more features of any other embodiment.

What is claimed is:

1. A method for manufacturing a monitoring unit, comprising:
providing one or more electrodes of the monitoring unit, wherein the one or more electrodes are configured to measure one or more physiological signals of the patient;
providing a signal processor configured to receive the one or more physiological signals; and
adding a hair-growth inhibitor to at least a portion of the one or more electrodes.

2. The method of claim 1, wherein the one or more electrodes comprises an adhesive portion and a gel portion.

3. The method of claim 2, wherein adding the hair-growth inhibitor to at least a portion of the one or more electrodes comprises adding the hair-growth inhibitor to the adhesive portion, wherein the adhesive portion comprises an adhesive configured to hold the one or more electrodes of the patient-portable remote monitoring unit into place on a chest of a patient.

4. The method of claim 2, wherein adding the hair-growth inhibitor to at least a portion of the one or more electrodes comprises adding the hair-growth inhibitor to the gel portion, wherein the gel portion comprises a material and a gel and is configured to form an electrical interface to measure the physiological signals.

5. The method of claim 4, wherein the gel is a silver chloride solution.

6. The method of claim 1, wherein adding the hair-growth inhibitor to at least a portion of the one or more electrodes comprises adding the hair-growth inhibitor to the adhesive portion and the gel portion.

7. The method of claim 1, further comprising creating inhibited electrodes.

8. The method of claim 7, further comprising applying a protective covering layer to the inhibited electrodes.

9. The method of claim 1, further comprising adding the hair-growth inhibitor to an external gel, wherein the external gel is added to the one or more electrodes of the patient-portable remote monitoring unit prior to being placed on a chest of a patient.

10. A monitoring unit to be worn by a patient, comprising:
one or more electrodes, comprising:
an adhesive portion of the one or more electrodes, wherein the adhesive portion of the one or more electrodes holds the one or more electrodes into place on a chest of the patient and comprises a hair-growth inhibitor; and
a gel portion of the one or more electrodes, wherein the gel portion comprises a material and a gel and is configured to form an electrical interface to measure the physiological signals; and
a signal processor configured to receive the one or more physiological signals.

11. The monitoring unit of claim 10, wherein the gel is a silver chloride solution.

12. The monitoring unit of claim 10, further comprising an external gel, wherein the external gel is added to the one or more electrodes of the patient-portable remote monitoring unit prior to being placed on a chest of a patient.

13. A monitoring unit to be worn by a patient, comprising:
one or more electrodes, comprising:
an adhesive portion of the one or more electrodes, wherein the adhesive portion of the one or more electrodes holds the one or more electrodes into place on a chest of the patient; and
a gel portion of the one or more electrodes, wherein the gel portion comprises a material and a gel and is configured to form an electrical interface to measure the physiological signals, and comprises a hair-growth inhibitor; and
a signal processor configured to receive the one or more physiological signals.

14. The monitoring unit of claim 13, wherein the chest is pre-treated with an additional hair-growth inhibitor.

15. The monitoring unit of claim 13, wherein the gel is a silver chloride solution.
